(19) United States
(12) Patent Application Publication
(10) Pub. No.: US 2006/0234270 A1
(43) Pub. Date: Oct. 19, 2006

(54) Normalization methods for gene expression analysis

(75) Inventor: Thomas M. Houts, Gilroy, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/379,223

(22) Filed: Apr. 18, 2006

Related U.S. Application Data
(60) Provisional application No. 60/672,687, filed on Apr. 18, 2005.

(51) Int. Cl. C12Q 1/68; C12P 19/34

(52) U.S. Cl. 435/6; 435/91.2

(57) ABSTRACT

The invention provides methods for obtaining an expected concentration for each of a plurality of housekeeping genes and using these values to obtain normalized concentrations for genes in an experimental sample using both external transcripts and internal housekeeping genes. A standard calibration curve is prepared using intensity values and concentrations of external transcripts. The standard calibration curve is used to obtain an expected concentration for each housekeeping gene. A calibration curve in an experimental sample is used to obtain preliminary concentrations for housekeeping genes and a correction factor is calculated from the expected concentrations. The correction factor is used to normalize values for genes in the experimental sample.

### RELATED APPLICATIONS

This application claims priority to U.S. Provisional Application No. 60/672,687, filed Apr. 18, 2005. The entire teachings of the above application are incorporated herein by reference in their entirety for all purposes.

### FIELD OF INVENTION

The present invention relates to methods and computer software products for normalizing gene expression measurements using exogenously added RNAs and endogenous RNAs.

### BACKGROUND OF THE INVENTION

In general, many normalization strategies for gene expression analysis are based on using housekeeping genes. Housekeeping genes are genes that are generally always expressed and coded for essential metabolic processes and substances. However, such strategies can become less effective when the genes are highly selected to represent primarily those which do change. Additionally, it can be difficult to find housekeeping genes which span the entire dynamic range of the system and which are not differentially expressed under the conditions of interest.

Another approach for normalization strategies for gene expression analysis includes using spiked-in external transcripts as normalization controls. However, variability in the concentration or quality of the unknown RNA external transcripts can cause systematic offsets between the external transcripts and the housekeeping genes.

This invention describes an approach for normalization based on using both external transcripts and housekeeping genes.

### SUMMARY OF THE INVENTION

In one aspect, the first step in the normalization process is to determine an expected concentration for a plurality of housekeeping genes by estimating and averaging their concentrations in a plurality of control samples. First, a RNA sample is obtained, for example, by extracting RNA from a tissue sample. Then external transcripts are added to the sample at known concentrations to generate a spiked sample. In a preferred aspect, 2 to 15 different external transcripts are added, each at a different concentration. The concentration range may be, for example, 0 to 130 attomoles per μg total RNA. The spiked sample is then amplified, labeled, and hybridized to an array to obtain a hybridization pattern. The hybridization pattern of the spiked sample is analyzed to obtain intensity values. A first calibration curve is prepared using the intensity values obtained for the external transcripts in the spiked sample on one axis and concentrations of the external transcripts on the other axis. The intensity values of the housekeeping genes are fit to the calibration curve of the spiked sample. From the first calibration curve, concentrations for each housekeeping gene are estimated and may be tabulated. This is repeated for a plurality of control samples that are matched samples, for example, a plurality of samples of the same tissue type from the same organism. Some of the common characteristics for tissue type include same organism, same organ, and same gender. Housekeeping genes that are highly expressed are found in the brain, kidney, liver, lung, muscle, prostate, and vulva. The estimated concentrations for each housekeeping gene are averaged to obtain an expected concentration for each housekeeping gene.

The second step in the normalization process is to obtain a normalized concentration of a target in an experimental sample. First, a nucleic acid sample is obtained from the experimental sample, for example, by extracting RNA from the experimental sample. Then a known amount of each of a plurality of external transcripts is added to the nucleic acid sample to generate a spiked sample. The spiked sample is then amplified, labeled, and hybridized to an array to obtain a hybridization pattern. The hybridization pattern of the spiked sample is analyzed to obtain intensity values. A calibration curve is prepared for the target of the experimental sample by plotting the intensity values of the external transcripts on one axis and concentrations of the external transcripts on the other axis. Intensity values from the housekeeping genes are compared to the external standards to obtain estimated concentrations for the housekeeping genes. Preliminary concentrations for other genes in the sample may also be estimated by comparison to the external standards. mA ratio (k) is calculated for each housekeeping gene by dividing the expected concentration of the housekeeping gene by the preliminary concentration of the housekeeping gene and a correction factor (K) is calculated by averaging the (k)s for a plurality of housekeeping genes. The intensity values for the remaining genes in the target are fit to the second calibration curve to obtain preliminary concentrations in the experimental sample. The preliminary concentrations for the genes in the target in the experimental sample are multiplied by the correction factor (K) to obtain normalized concentrations in the experimental sample.

In another aspect of the invention, a computer software product is developed for obtaining a normalized concentration for a target in an experimental sample. A computer program code for the calibration curve is prepared for the experimental sample by plotting the intensity values of the external transcripts on one axis and concentrations of the external transcripts on the other axis. A computer program code for intensity values from the housekeeping genes are fit to the calibration curve and from the calibration curve, preliminary concentrations of the housekeeping genes are estimated. A computer program code for a ratio (k) is calculated for each housekeeping gene by dividing the expected concentration of the housekeeping gene by the preliminary concentration of the housekeeping gene and a correction factor (K) is calculated by averaging the (k)s for a plurality of housekeeping genes. A computer program code for the intensity value is fit to the second calibration curve to obtain a preliminary concentration in the experimental sample. A computer program code for the preliminary concentration of the experimental sample is multiplied by the correction factor (K) to obtain a normalized concentration for the experimental sample.

In another aspect of the invention, a computer readable medium having computer executable instructions is developed for obtaining a normalized concentration for a target in an experimental sample. The calibration curve is prepared for the experimental sample by plotting the intensity values of the external transcripts on one axis and concentrations of the external transcripts on the other axis. The intensity values from the housekeeping genes are fit to the calibration curve and from the calibration curve, preliminary concentrations of the housekeeping genes are estimated. The ratio (k) is calculated for each housekeeping gene by dividing the expected concentration of the housekeeping gene by the preliminary concentration of the housekeeping gene and a correction factor (K) is calculated by averaging the (k)s for a plurality of housekeeping genes. The intensity value is fit to the second calibration curve to obtain a preliminary concentration in the experimental sample. The preliminary concentration of the experimental sample is multiplied by the correction factor (K) to obtain a normalized concentration for the experimental sample.

### DETAILED DESCRIPTION

a) General

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GENECHIP. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. The sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu et al., Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young et al., P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of a method according to the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal et al., Introduction to Computational Biology Methods (PWS Publishing Company, Boston, 1997); Salzberg et al., (Ed.), Computational Methods in Molecular Biology, (Elsevier, Amsterdam, 1998); Rashidi et al., Bioinformatics Basics: Application in Biological Science and Medicine (CRC Press, London, 2000) and Ouelette et al. Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), 10/065,856, 10/065,868, 10/328,818, 10/328,872, 10/423,403, and 60/482,389.

It is well known to one of the skill in the art that external and internal transcripts can be used to normalize gene expression analysis. External and internal transcripts are the subject of numerous publications including, e.g. U.S. Pat. No. 6,308,170, U.S. Pat. No. 6,841,348, U.S. Patent Publication 20010044132, U.S. Patent Publication 20030148339, U.S. Patent Publication 20030175726, European Patent Publication 1490512, and European Patent Publication 1386009 all incorporated herein by reference. Housekeeping genes, or maintenance genes, are those genes constitutively expressed to maintain cellular function (See, Watson et al. Molecular Biology of the Gene, Vol. 1, 1965). Previously many genes have been reported as putative housekeeping genes. The genes previously reported were identified by conventional methods and the putative housekeeping role of the gene product is an incidental observation (Duhig, T. et al. Genomics, 52(1) 72-78, 1998; Hampsey, M., Microbiol. Mol. Biol. Rev. 62(2):465-503, 1998; May et al. Mol. Biol. Med., 7(5):405-421, 1990; Milner C., et al. Bioessays, 14(8):565-571, 1992; Rifkind R. A., et al. Ann. Immunol. 127:887-893, 1976; Robertson H. A., et al., Biochem. Cell Biol., 70(9): 729-737, 1992; Russo-Marie, F., J Neuroimmunol, 40(2-3):281-286, 1992; Strehler, B. L. et al. Mech. Aging Dev. 14(1-2) 15-38, 1980; and Yamamoto, T., Y. Matsui et al., Gene 80 2:337-343, 1989).

Recently, massive parallel gene expression monitoring methods have been developed to monitor the expression of a large number of genes using nucleic acid array technology which was described in detail in, for example, U.S. Pat. Nos. 5,871,928, 5,800,992 and 6,040,138; De Saizieu, et al., 1998, Nature Biotechnology, 16:45-48; Wodicka et al., 1997, Nature Biotechnlogy 15:1359-1367; Lockhart et al., 1996, Nature Biotechnology 14:1675-1680; Lander, 1999, Nature-Genetics, 21(suppl.), at 3.

b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, which are typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM and most preferable at about 100 mM. Hybridization temperatures can be as low as 5° C., but are typically greater than about 37° C., and most preferable at 45°-50° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 100 mM MES, 1M Na+, 20 mM EDTA, 0.01% Tween-20 and a temperature of about 45° C. For stringent conditions, see, for example, Affymetrix's GeneChip® Expression Analysis which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

Hybridizing specifically to: refers to the binding, duplexing, or hybridizing of a molecule only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

The methods disclosed herein may also be used for analysis of protein levels using, for example, protein arrays. The levels of the protein products of housekeeping genes may be measured and used to normalize the levels of other proteins. Similarly, known proteins may be spiked into the sample at known levels and used to generate a calibration curve.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptor is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “Probe Target Pair” is formed when two macromolecules have combined through molecular recognition to form a complex.

Housekeeping genes or maintenance genes are a class of genes used in normalization of gene expression analysis that have a relatively constant expression level among the samples compared and code for essential metabolic processes. Housekeeping genes provide suitable reference genes for an individual relative quantification approach based on the estimated relative abundance of the target gene in the sample. Genes which contain housekeeping function may exhibit variable expression levels from one tissue to another. Amplification of housekeeping genes verifies that the target nucleic acid and reaction components were of acceptable quality. Examples of housekeeping genes commonly considered to have a housekeeping function include β-actin, GAPDH, glyceraldehyde-3-phosphate dehydrogenase (G3PDH), albumin, actins, tubulins, cyclophilin, hypoxantine phosphoribosyltransferase (HRPT), L32. 28S and 18S rRNAs. Housekeeping genes are found to be highly expressed in the following organs: brain, kidney, liver, lung, muscle, prostate, placenta, stomach, testes, ovary, myometrium, esophagus, endometrium, cervix, colon, and vulva. Some functions of housekeeping genes include gene expression/transcription, signaling/communications, defense apoptosis, cell division, metabolism, structure/motility, ribosomal protein, and protein expression/transcription.

External transcripts or exogenous transcripts are transcripts that are added or spiked-in as calibration or normalization controls for target preparations. In a preferred embodiment, the transcripts are RNA. An example of external transcripts is taken from yeast intergenic regions (YIR) mRNAs which correspond to a particular dynamic range and ratio controls and each mRNA is represented at a different specified concentration and ratio. Another example of external transcripts is taken from bacterial mRNAs which correspond to a particular dynamic range and ratio controls and each mRNA is represented at different specified concentration and ratio.

Correction factor is defined as the ratio of expected concentration of a housekeeping gene of a biological sample to a preliminary concentration of a housekeeping gene of an experimental sample and averaging the plurality of ratios.

Extrapolated straight line is a straight line which is projected, extended, or expanded (known data or experience) into an area not known or experienced so as to arrive at a usually conjectural knowledge of the unknown area. It can also mean to predict by projecting past experience or known data.

Mean is defined as the sum of all values divided by their number. It is customary to differentiate between the mean of the population which is the mean of all values in the population considered and the mean of a sample which is the mean of the subset of the population members included in the sample under consideration. The sample mean is usually reported to one more decimal place than the data. Additionally, the sample mean is measured in the same measurement units as the data.

Median is the value situated in the middle of the ordered list of measurements. The median can also be described as the value that is lower than 50% of the data and higher than the other 50% of it. It can also be described as an estimate of the 50-th percentile.

Normalization is a type of pre-processing done to account for systematic differences across the data sets. For example, normalization is modifying values in order to compensate for different dye efficiency in a two channel microarray experiment using cy3 and cy5.

Parabolic curve is a plane curve generated by a point moving so that its distance from a fixed point is equal to its distance from a fixed line.

Range is the interval between the smallest and the largest measurement in a group. A wider range will indicate a larger variability than a narrower range.

Aliquot means a fraction of something. Aliquot also means contained an exact number of times in something or exact divisor or factor of a quantity.

c) Normalization Methods

Methods for normalization for gene expression analysis are frequently based on endogenous transcripts, for example, “housekeeping” or “maintenance” genes. Housekeeping genes, for example, may be used as internal standards for microarray experiments where thousands to tens of thousands of genes are analyzed simultaneously. See, for example, Hsiao et al., Physiol Genomics 7(2):97-104 (2001) and Warrington et al., Physiol Genomics 2(3):143-7 (2000). See also, U.S. Pat. No. 6,884,578. The use of housekeeping genes for normalization works most effectively when many transcripts are analyzed and relatively few are expected to change. However, these methods can become less effective when fewer transcripts are analyzed, for example, when the genes to be analyzed are a subset of genes that are selected to represent primarily those that are expected to change. Analysis of smaller number of transcripts, for example, 100 to 2,000, may be the method of choice for diagnostic applications. Additionally, it can be difficult to find a set of housekeeping genes which span the entire dynamic range of the system and are not differentially expressed under the conditions of interest.

Another approach for normalization strategies for gene expression analysis includes using spiked-in external transcripts as normalization controls. However, variability in the concentration or quality of the unknown RNA external transcripts can cause systematic differences between the external transcripts and the housekeeping genes.

In a preferred embodiment of the disclosed methods, normalization is based on both external transcripts and internal housekeeping genes. The methods provide a linear or non-linear normalization over the range of the system. Additionally, in some aspects the set of housekeeping genes that is used is smaller than the set that may be required for methods based entirely on internal normalization. In preferred aspects the external control transcripts may be used for multiple purposes, for example, the modeling of error as a function of signal, the generation of a precision profile, and improvement of data transferability from one experimental system to another.

In one embodiment the steps of the method are as follows. Obtain external RNA transcripts to use as calibrators for RNA concentration. Arrays are designed to include probes to each of the RNAs in the set. Preferably the RNAs selected are ones that do not naturally occur in the samples to be analyzed so that the probes that are complementary to the RNAs do not cross hybridize with other sequences that are present in the samples. Preferably the set includes enough different RNAs to cover a range of concentrations from below the detection limit to higher than the most abundant transcript in the experimental sample. Sets of RNAs that can be used for external controls according to the method are commercially available, for example, Lucidea Universal ScoreCard (Amersham Biosciences) and standard sets under development by the External RNA Controls Consortium (ERCC). See Baker et al., Nat Methods 2(10):731-4 (2005).

In a preferred embodiment, the external transcripts are RNA. The concentration range preferably starts from below a detection limit and ends at a saturation point. In another embodiment, the known concentration range is in attomoles per μg of RNA, for example, 0-130 attomoles per 1 μg RNA. In yet another embodiment, the known concentration range is in μmoles per μg of RNA.

External transcripts are preferably not found in the sample being analyzed. For example, the external transcripts to be used in analysis of a mammalian sample may come from another organism such as yeast or bacteria. The RNA may also be synthetic sequence not present in any known genome. For example, they may be antigenomic sequences as described in Taylor, J. M. et al., Fox Chase Cancer Center 2003 Scientific Report; Wichlacz, A. et al., Nucleic Acids Research, 2004, 32(3): e39; U.S. Pat. Nos. 6,719,979, 6,497,873, and 6,376,236. In another preferred embodiment, at least one of the external transcripts is from a yeast intergenic region (YIR). In yet another embodiment, at least one of the external transcripts is from bacterial controls. Tag sequences may also be used as synthetic external controls. RNA spikes from Tag genes spikes have been used as exogenous controls in quantitative RT-PCR experiments. These spikes can be used to normalize quantitative RT-PCR to aid in determine absolute transcript levels. In addition, the Tag gene spikes can also allow direct comparisons between the microarray and RT-PCR results or between different types of microarrays, for example, spotted arrays versus oligonucleotide arrays synthesized using photolithography. The universal absence of the synthetic genes will also allow comparisons between different sample types such mouse, human, and bacteria. See U.S. Patent Publication 20040175719 which is hereby incorporated by reference.

The next step is to obtain one or more RNA samples as controls, for example, by extracting RNA from a tissue sample. The nucleic sample may be total RNA, a cDNA sample, poly(A) RNA, an RNA sample depleted of one or more RNAs, for example, an RNA sample depleted of rRNA or an amplification product of RNA. In one aspect the sample, is from a mammal, for example, a human, a rat or a mouse. The sample may be isolated from a tissue, including, for example, blood, lung, heart, kidney, pancreas, prostate, testis, uterus, brain, or skin.

Add the selected external transcripts to the sample at known concentrations to generate a spiked sample. In a preferred aspect between 2 and 15 different external transcripts are added, each at a different concentration. In another aspect, two or more different transcripts may be added at the same concentration for redundancy. The concentration range may be, for example, 0 to 130 attomoles per μg total RNA (“0” may be a transcript that is not added to the sample but a probe set for the transcript is included on the array and should give a background intensity). In one example, external transcripts are spiked in at the following concentrations (in attomoles per μg total RNA): 0, 0.1124, 0.225, 0.45, 0.9, 1.8, 3.6, 7.2, 14.5, 29, 58 and 116 in another example the spikes are at 0, 0.08, 0.24, 0.76, 2.40, 7.60, 24.03, 75.99, 120.16 and 125.00. If fewer transcripts are to be spiked in they should cover the high and low ends of the range, for example, 0.1, 62.5 and 125. The spiked sample is then amplified, labeled, and hybridized to an array to obtain a hybridization pattern. The external spiked-in transcripts are processed along with the sample. The sample may be hybridized, for example, to a Human Genome U133 array from Affymetrix, or to an Affymetrix All Exon Array.

The hybridization pattern is analyzed to obtain intensity values for the external transcripts. A calibration curve is prepared for each sample using the intensity values obtained for the external transcripts and the known concentrations of the external transcripts. The intensity values of the housekeeping genes are compared to the calibration curve of the external transcripts. The curve can be linear but may also be a linear extrapolation, sigmoidal, or parabolic in shape. From the external transcript calibration curve, concentrations for each housekeeping gene are estimated. In a preferred aspect, this is repeated for a two or more control samples that are matched to the experimental sample in one or more physiological or biological states, for example, two or more samples of the same tissue type from the same organism. Some of the common characteristics for tissue type include, for example, same organism, for example, human or mouse, same organ, for example, heart or lung, same gender, same disease state, same age, and same race. The estimated concentrations for each housekeeping gene are averaged over the multiple samples to obtain an expected concentration for each housekeeping gene. The expected concentration for each housekeeping gene may be the mean or median observed concentration for that housekeeping gene.

The nucleic sample may be total RNA, a cDNA sample, poly(A) RNA, an RNA sample depleted of one or more RNAs, for example, an RNA sample depleted of rRNA or an amplification product of RNA. In one aspect the sample, is from a mammal, for example, a human, a rat or a mouse. The sample may be isolated from a tissue, including, for example, blood, lung, heart, kidney, pancreas, prostate, testis, uterus, brain, or skin.

The expected concentrations obtained from the control samples for the housekeeping genes are used to obtain normalized concentrations for transcripts in an experimental sample. First, a nucleic acid sample is obtained from the experimental sample. Then a known amount of each of the external transcripts is added to the experimental nucleic acid sample to generate a spiked sample. The spiked sample is then amplified, labeled, and hybridized to an array to obtain a hybridization pattern using the same procedures used for the control samples. The hybridization pattern is analyzed to obtain intensity values for the external transcripts, the housekeeping genes and other genes of interest. A calibration curve is prepared for the experimental sample using the intensity values of the external transcripts and their known concentrations. Regression analysis is performed using the calibration curve and the intensity values from all genes of interest, including the housekeeping genes, to obtain estimated preliminary concentrations for all genes. A ratio (k) is calculated for each housekeeping gene by dividing the expected concentration of the housekeeping gene (from the control samples) by the preliminary concentration of the housekeeping gene (in the experimental sample). The ratios (k) from a plurality of housekeeping genes may be combined (e.g., averaged) to obtain a correction factor (K), reflecting systematic variation from the expected concentration. The preliminary concentration for the remaining genes from the experimental sample is multiplied by the correction factor (K) to obtain a normalized concentration in the experimental sample for all genes of interest.

The correction factor (K) may be the mean of all ratios of expected concentration to preliminary concentration or the median of all ratios of expected concentration to preliminary concentration. In a preferred embodiment the correction factor is at least 0.01 or at least 0.1.

The method may also be implemented as a computer software product for obtaining a normalized concentration target in an experimental sample. First, a computer program code is used for preparing a standard calibration curve with intensity values of the external transcripts on one axis and concentrations of external transcripts on another axis. A computer program code is used for fitting a line through a plurality of points of the experimental calibration curve. A computer program code is used to fit the intensity values from each housekeeping gene to the standard calibration curve. A computer program code is used for calculating a preliminary concentration for each housekeeping gene and to determine a ratio of expected concentration to preliminary concentration of a housekeeping gene and averaging the plurality of ratios to obtain a correction factor. A computer program code is used for fitting the intensity value of the target onto a second calibration curve to obtain a preliminary concentration for the target in an experimental sample. A computer program code is used for multiplying the preliminary concentration of the target with the correction factor to obtain a normalized concentration of the target in the experimental sample.

In a preferred embodiment, a computer software product is used where the correction factor is the mean of all ratios of expected concentration to preliminary concentration. In a preferred embodiment, a computer software product is used where the correction factor is the median of all ratios of expected concentration to preliminary concentration. In a preferred embodiment, a computer software product is used wherein the correction factor is at least 0.01. In another aspect of the invention, a computer readable medium having computer executable instructions for obtaining a normalized concentration target in an experimental sample is described. A computer is used to obtain an experimental calibration curve using the intensity values of the external transcripts and the concentrations of external transcripts. The computer is used to fit the intensity values from each housekeeping gene onto the calibration curve and to calculate a preliminary concentration for each housekeeping gene. A ratio of expected concentration of a housekeeping gene to the preliminary concentration of a housekeeping gene is calculated and an average of the ratios is calculated to obtain a correction factor. The intensity value of the target is fit onto a second calibration curve to obtain a preliminary concentration for the target in an experimental sample. Then the preliminary concentration of the target is multiplied by the correction factor to obtain a normalized concentration of the target in the experimental sample.

In a preferred embodiment, a computer readable medium is used wherein the experimental calibration curve is sigmoidal or S-shaped. In another embodiment, a computer readable medium is used wherein the experimental curve is parabolic. In another embodiment, a computer readable medium is used wherein the experimental calibration curve is an extrapolated straight line. In a preferred embodiment, a computer readable medium is used where the correction factor is the mean of all ratios of expected concentration to preliminary concentration. In a preferred embodiment, a computer readable medium is used where the correction factor is the median of all ratios of expected concentration to preliminary concentration. In a preferred embodiment, a computer readable medium is used wherein the correction factor is at least 0.01, at least 0.1, at least 1.0, at least 10, at least 100 or at least 1000.

Spiking can serve as a control for various aspects of the assay process such as variations in sample preparation, hybridization conditions, and array quality. Arrays may be designed to include probes to detect a variety of different external transcripts. A spike set is being developed by the External RNA Controls Consortium (ERCC). The External RNA Controls Consortium (ERCC) is composed of representatives from the public, private, and academic sectors, working together to produce external RNA spike-in controls that will be useful for sample control on a variety of microarray platforms and by RT-PCR. These platform-independent control materials will enhance performance evaluation of reproducibility, sensitivity, and robustness in gene expression analysis and in a preferred aspect may be used as external controls in the methods disclosed herein. The ERCC plans to develop a set of 100 well characterized clones comprised of random unique sequence as determined by sequence comparison to mouse, rat, human, drosophila, E. coli, mosquito sequence databases, as well as well characterized Bacillus subtillus and Arabadopsis thaliana clones.

Additionally, external RNA transcripts that may be used for the disclosed methods are commercially available, for example, from GE Healthcare (Lucidea Universal ScoreCard). The Lucidea Universal ScoreCard is a set of 23 unique microarray controls that can be used with samples from most species and with any microarray platform. The controls are artificial genes that generate pre-determined signal intensities that do not change across samples or experiments. With Lucidea Universal ScoreCard, microarray analysis is not dependent on relative quantification. The controls may be used to generate a calibration curve for determining limits of detection, linear range, and data saturation, and they can be used as universal references for validating and normalizing microarray data.

The dynamic range and ratio controls can be artificial genes which are selected to ensure no cross-hybridization with each other. The spike-mixes include calibration and ratio controls in which the calibration controls span multiple orders of magnitude and the ratio controls can range from low to high. Additionally, there are as many as eight ratio controls at two expression levels.

YIR controls mRNAs for the spike mix can be generated by in vitro transcription. In vitro transcription are generated by amplification of primers that are designed to introduce a T7 RNA polymerase promoter on the 5′ end and d(T) tail on the 3′ end of the PCR product. The transcription products are purified using a Qiagen kit. Bacterial mRNAs may also be used for external transcripts.

The hybridization conditions between probe and target should be selected such that the specific recognition interaction, i.e., hybridization, of the two molecules, is both sufficiently specific and sufficiently stable. See, e.g., Hames and Higgins (1985) Nucleic Acid Hybridisation: A Practical Approach, IRL Press, Oxford. These conditions will be dependent both on the specific sequence and often on the guanine and cytosine (GC) content of the complementary hybrid strands. The conditions may often be selected to be universally equally stable independent of the specific sequences involved. This typically will make use of a reagent such as an alkylammonium buffer. See, Wood et al. (1985) “Base Composition-independent Hybridization in Tetramethylammonium Chloride: A Method for Oligonucleotide Screening of Highly Complex Gene Libraries,” Proc. Natl. Acad. Sci. USA, 82:1585-1588; and Krupov et al. (1989) “An Oligonucleotide Hybridization Approach to DNA Sequencing,” FEBS Letters, 256:118-122; each of which is hereby incorporated herein by reference. An alkylammonium buffer tends to minimize differences in hybridization rate and stability due to GC content. By virtue of the fact that sequences then hybridize with approximately equal affinity and stability, there is relatively little bias in strength or kinetics of binding for particular sequences.

Temperature and salt conditions along with other buffer parameters should be selected such that the kinetics of renaturation should be essentially independent of the specific target subsequence or oligonucleotide probe involved. In order to ensure this, the hybridization reactions will usually be performed in a single incubation of all the substrate matrices together exposed to the identical same target probe solution under the same conditions. The hybridization conditions will usually be selected to be sufficiently specific such that the fidelity of base matching will be properly discriminated. Of course, control hybridizations should be included to determine the stringency and kinetics of hybridization. See for example, U.S. Pat. No. 5,871,928 which is hereby incorporated in its entirety for all purposes. Another factor that can be adjusted to increase the ability of targets to hybridize to probes, is the use of nucleic acid analogs or PNAs in the probes.

They can be built into the probes to create a more uniform set of hybridization conditions across the entire array. See U.S. patent application Ser. No. 08/630,427 which is hereby incorporated by reference in its entirety for all purposes.

Samples are then washed and stained using a robotic liquid handling machine such as the GeneChip® Fluidic Station 400 or the GeneChip® Fluidic Station 450/250 (Affymetrix, Inc., Santa Clara, Calif.). Fluidics stations have been described in, for example, U.S. patent application Ser. No. 08/624,133 now abandoned and Ser. No. 09/070,689 now U.S. Pat. No. 6,114,122. Finally, samples are placed on an automated loader which interfaces with a scanner. Scanners have been described in, for example, U.S. Pat. Nos. 5,578,832, 5,834,758 and 5,837,832, U.S. patent application Ser. Nos. 08/456,598, 09/238,131 now U.S. Pat. No. 6,225,625 now U.S. Pat. No. 6,270,644, Ser. No. 08/856,642 now U.S. Pat. No. 5,981,956, Ser. No. 09/295,214 now U.S. Pat. No. 6,207,960, Ser. No. 08/456,782 now abandoned, Ser. No. 08/999,188 now U.S. Pat. No. 6,491,811, U.S. Provisional Patent Application No. 60/106,397 and European Patent No. 97925605 each of which is hereby incorporated by reference in its entirety for all purposes.

The results are then analyzed using a computer program. Computer programs for the analysis of hybridization patterns on arrays have been described in, for example, U.S. Pat. Nos. 5,733,729, and 5,795,716, U.S. patent application Ser. No. 09/309,328 now U.S. Pat. No. 6,197,516, Ser. No. 09/020,743 now U.S. Pat. No. 6,420,108, Ser. No. 08/531,137 now U.S. Pat. No. 5,974,164, Ser. Nos. 09/158,765, 08/584,754, 09/049,805, 08/828,952, 08/948,896 and U.S. Provisional Patent Application Nos. 60/033,053 and 60/085,118 each of which is incorporated by reference in its entirety for all purposes.

Specific examples of housekeeping genes include the following genes which may be used in the normalization process disclosed herein: Homo sapiens U5 snRNP-specific protein (220 kD), ortholog of S. cerevisiae Prp8p (PRP8) gB: NM_{—}006445.1, Homo sapiens calpain 4, small subunit (30K) (CAPN4) gB: NM_{—}001749.1, Homo sapiens ribosomal protein L35 (RPL35) gB: NM_{—}007209.1, Homo sapiens ribosomal protein L28 (RPL28) gb:NM_{—}000991.1, Homo sapiens eukaryotic translation initiation factor 4 gamma, 2 (EIF4G2) gb:NM_{—}001418.1, Homo sapiens eukaryotic translation initiation factor 3, subunit 7 (zeta, 6667 kD) (EIF3S7) gb:NM_{—}003753.1, Homo sapiens RNA-binding protein regulatory subunit (DJ-1) gb:NM_{—}007262.1, Homo sapiens signal recognition particle 14 kD (homologous Alu RNA-binding protein) (SRP14) gb:NM_{—}003134.1, Human rab GDI mRNA gb:D13988.1, Homo sapiens GDP dissociation inhibitor 2 (GDI2) gb:NM_{—}001494.2, Homo sapiens ribosomal protein L11 (RPL11) gb:NM_{—}000975.1, Homo sapiens ADP-ribosylation factor 3 (ARF3) gb:NM_{—}001659.1, Homo sapiens ribosomal protein L21 (gene or pseudogene) (RPL21) gb:NM_{—}000982.1, Homo sapiens ribosomal protein L24 (RPL24) gb:NM_{—}000986.1, Homo sapiens heterogeneous nuclear ribonucleoprotein C(C1C2) (HNRPC) gb:NM_{—}004500.1, Homo sapiens neural precursor cell expressed, developmentally down-regulated 5 (NEDD5) gb:NM_{—}004404.1, Homo sapiens heterogeneous nuclear ribonucleoprotein A1 (HNRPA1) gb:NM_{—}002136.1, Homo sapiens ribosomal protein S27a (RPS27A) gb:NM_{—}002954.1, Homo sapiens ribosomal protein S13 (RPS13) gb:NM_{—}001017.1, Homo sapiens Finkel-Biskis-Reilly murine sarcoma virus (FBR-MuSV) gb:NM_{—}001997.1, Homo sapiens TAR DNA binding protein (TARDBP) gb:NM_{—}007375.1, Homo sapiens cofilin 1 (non-muscle) (CFL1) gb:NM_{—}005507.1, Homo sapiens ribosomal protein L18 (RPL18) gb:NM_{—}000979.1, Homo sapiens eukaryotic translation initiation factor 3, subunit 5 (epsilon, 47 kD) (EIF3S5) gb:NM_{—}003754.1, Homo sapiens ribosomal protein S5 (RPS5) gb:NM_{—}001009.1, Homo sapiens ribosomal protein L27 (RPL27) gb:NM_{—}000988.1, Homo sapiens ribosomal protein L34 (RPL34) gb:NM_{—}000995.1, Homo sapiens asparaginyl-tRNA synthetase (NARS) gb:NM_{—}004539.2, Homo sapiens GTT1 protein (GTT1) gb:NM_{—}020151.1, Homo sapiens ribosomal protein L19 (RPL19) gb:NM_{—}000981.1, Homo sapiens solute carrier family 25 (mitochondrial carrier; phosphate carrier), member 3 (SLC25A3), nuclear gene encoding mitochondrial protein, transcript variant 1b gb:NM_{—}002635.1, Homo sapiens ribosomal protein S11 (RPS11) gb:NM_{—}001015.1, Homo sapiens ribosomal protein L9 (RPL9) gb:NM_{—}000661.1, Homo sapiens DEADH (Asp-Glu-Ala-AspHis) box polypeptide 5 (RNA helicase, 68 kD) (DDX5) gb:NM_{—}004396.2, Homo sapiens ribosomal protein L6 (RPL6) gb:NM_{—}000970.1, Homo sapiens hypothetical protein (HSA011916) gb:NM_{—}015343.1, Homo sapiens ribosomal protein L10a (RPL10A) gb:NM_{—}007104.2, Homo sapiens heterochromatin-like protein 1 (HECH) gb:NM_{—}016587.1, Homo sapiens ribosomal protein L17 (RPL17) gb:NM_{—}000985.1, Homo sapiens proteasome (prosome, macropain) subunit, beta type, 2 (PSMB2) gb:NM_{—}002794.1, Homo sapiens GAP-associated tyrosine phosphoprotein p62 (Sam68) (SAM68) gb:NM_{—}006559.1, Homo sapiens HLA-B associated transcript-1 (D6S81E) gb:NM_{—}004640.1, Homo sapiens hypothetical protein (HSPC117) gb:NM_{—}014306.1, Homo sapiens enhancer of rudimentary (Drosophila) homolog (ERH) gb:NM_{—}004450.1, Homo sapiens splicing factor, arginineserine-rich 9 (SFRS9) gb:NM_{—}003769.1, Homo sapiens ATP-binding cassette, sub-family F (GCN20), member 1 (ABCF1) gb:NM_{—}001090.1, Homo sapiens defender against cell death 1 (DAD1) gb:NM_{—}001344.1, Homo sapiens YY1 transcription factor (YY1) gb:NM_{—}003403.2, Homo sapiens jumping translocation breakpoint (JTB) gb:NM_{—}006694.1, Homo sapiens histone acetyltransferase (HBOA) gb:NM_{—}007067.1, Homo sapiens zinc finger protein 146 (ZNF146) gb:NM_{—}007145.1, Homo sapiens squamous cell carcinoma antigen recognised by T cells (SART1) gb:NM_{—}005146.1, Homo sapiens interleukin enhancer binding factor 2, 45 kD (ILF2) gb:NM_{—}004515.1, Homo sapiens sperm associated antigen 7 (SPAG7) gb:NM_{—}004890.1, Homo sapiens zinc finger protein 259 (ZNF259) gb:NM_{—}003904.1, Homo sapiens TATA box binding protein (TBP)-associated factor, RNA polymerase II, H, 30 kD (TAF2H) gb:NM_{—}006284.1, Homo sapiens nuclear DNA-binding protein (C1D) gb:NM_{—}006333.1, Homo sapiens non-POU-domain-containing, octamer-binding (NONO) gb:NM_{—}007363.2, Homo sapiens, Similar to U5 snRNP-specific protein, 200 kDa (DEXH RNA helicase family), clone MGC:2580 gb:BC001417.1, Homo sapiens, ras homolog gene family, member A, clone MGC:2330 gb:BC001360.1, Homo sapiens, RNA-binding protein S1, serine-rich domain, clone MGC:1125 gb:BC001659.1, Homo sapiens, Similar to ribosomal protein S24, clone MGC:8595 gb:BC000523.1, Homo sapiens ribosomal protein L30 gb:L05095.1, Homo sapiens, nucleophosmin (nucleolar phosphoprotein B23, numatrin), clone MGC:8463 gb:BC002398.1, Homo sapiens isolate Liv chaperone protein HSP90 beta (HSP90BETA) gb:AF275719.1, Homo sapiens chondrosarcoma-associated protein 2 (CSA2) gb:AF182645.1, Human DNA sequence from clone RP3-429G5 on chromosome 6q21-22.1 gb:AL078596, Human calnexin gb:M94859.1, KIAA0156 gene product gb:AI656011, Homo sapiens, hypothetical protein, clone MGC:782 gb:BC001393.1, splicing factor 30, survival of motor neuron-related gb:BF224259, Homo sapiens M4 protein deletion mutant gb:AF061832.1, Homo sapiens hnRNP-C like protein gb:M94630.1, Homo sapiens CAG-isl 7 gb:U16738.1, Homo sapiens, guanylate kinase 1, clone MGC:10618 gb:BC006249.1, Homo sapiens, hypothetical protein MGC2749 gb:BC006479.1, Human mRNA for ornithine decarboxylase antizyme gb:D87914.1, Homo sapiens, ATPase, H+ transporting, lysosomal (vacuolar proton pump) 21 kD, clone MGC:4498 gb:BC005876.1, Homo sapiens lysyl-tRNA synthetase gb:AF285758.1, H3 histone, family 3A gb:AI955655, ribosomal protein S6 gb:BE741754, ribosomal protein S7 gb:AI805587, ubiquitin specific protease 22 gb:AA621731, small acidic protein gb:BE748698, Homo sapiens transcription elongation factor B (SIII), polypeptide 2 (18 kD, elongin B) (TCEB2) gb:NM_{—}007108.1, cytochrome c oxidase subunit IV gb:AA854966, Homo sapiens cDNA gb:AK024976.1, ribosomal protein L12 DEF=Homo sapiens cDNA gb:AK026491.1, ribosomal protein L4 gb:AI953886, farnesyltransferase, CAAX box, alpha gb:BG168896, ribosomal protein S25 gb:AA888388, ribosomal protein L37 gb:BF216701, histidine triad nucleotide-binding protein gb:N32864, eukaryotic translation elongation factor 2 gb:AI004246, ribosomal protein S10 gb:AA320764, ATPase, H+ transporting, lysosomal (vacuolar proton pump) 9 kD gb:AI862255, heterogeneous nuclear ribonucleoprotein K gb:AI701949, anaphase-promoting complex subunit 5 gb:T33068, and Human DNA sequence from clone RP11-486O22 on chromosome 10 Contains the 3part of a gene for KIAA1128 protein, a novel pseudogene, a gene for protein similar to RPS3A (ribosomal protein S3A), ESTs, STSs, GSSs and CpG islands gb:AL356115. These housekeeping genes are standard and be used in the normalization process.

The number of housekeeping genes to be analyzed is preferably 10 or more, 20 or more, 50 or more or 100 or more genes. Housekeeping genes are generally selected so that they are expressed at about the same level in the tissue or tissues of interest. Housekeeping genes should be selected so that their expression does not vary or varies little in the tissue of interest. The method further comprises the step of indicating that the gene is a housekeeping gene if the expression is approximately the same in all the tissues of interest or in a subset of the tissues of interest.

The term tissue, as used herein, is intended to describe a biological material from an organism. Therefore, an organ (or a homogenate of the organ), such as the liver or kidney, may be referred to as a tissue. The methods are most suitable for simultaneously detecting a large number of housekeeping genes. When it is used for simultaneous determination of a large number of housekeeping genes, the method includes the step of simultaneous monitoring of the expression of a large number of genes. Methods for monitoring a large number of genes are well known in the art and are described, for example, in the background section. In some embodiments, the expression of a gene in a number of tissues is measured. The gene is considered as expressed at the same level if it is expressed in all the tissues at levels within ten fold, preferably within fourfold and more preferably within two fold. In some embodiments, a gene is considered as expressed at the same level if it is expressed in all tissues with no statistically significant difference. In the example that follows, genes were considered as expressed at the same level if they were expressed in all seven tissues at levels within four fold. For most genes differences less than four fold are not biologically significant but there is not enough data to conclude that a five or six fold difference is more biologically significant than a three or four fold difference (Cho, R. J. et al., Molecular Cell, 2:65-73, 1998; Creanor, J., et al., Journal of Cell Science 207-215, 1986; Klevecz, R. R. The Scientist 22-24, 1999; Klevecz, R. R. et al., International Review of Cytology, 86:97-128, 1984). For a subset of genes it is likely that small differences have biological relevance such as the genes encoding proteins that function differently when bound to high affinity versus low affinity receptors or gene products triggering cellular cascades (Merchav, S., J. Pediatr. Endocrin. Metab. 11(6):677-685, 1998; Skerry, T. M., J. Bone Miner Metab. 17(1):66-70, 1999).

### EXAMPLE

1. Sample Preparation

All samples were prepared from pools of human adult poly(A) RNA purchased from Clontech (Palo Alto, Calif.). The tissues screened are listed followed by the number of tissues pooled and the Clontech catalog number in parenthesis. Heart, 3 (6533-1), brain, 5 (6516-1), lung, 5 (6524-1), kidney, 8 (6538-1), pancreas, 10 (6539-1), uterus, 10 (6537-1), testis, 19 (6535-1). Poly(A) RNA was amplified and labeled with biotin following the procedure described by Wodicka et al., 1997.sup.(32). First strand cDNA synthesis was carried out at 42° C. for 60 minutes. The amplified cRNA (target) was purified on an affinity resin (RNeasy, Qiagen) and quantitated.

2. Fragmentation, Array Hybridization and Scanning

Labeled target was fragmented by incubation at 94° C. for 35 minutes in the presence of 40 mM Tris-acetate pH 8.1, 100 mM potassium acetate, and 30 mM magnesium acetate. The hybridization solution consisted of 15 ug fragmented cRNA, 10 mg/ml sonicated herring sperm DNA in buffer containing 100 mM MES, 1 m[Na.sup.+], 20 mM EDTA, 0.01% Tween 20 (MES). The hybridization mixture was heated to 99° C. for 5 min. followed by incubation at 45° C. for 10 min. before injection of the sample into the probe array cartridge. All hybridizations were performed in duplicate and were carried out at 45° C. for 16 hours with mixing on a rotisserie at 60 rpm. Following hybridization, the solutions were removed, arrays were rinsed with 1×MES (100 mM MES, 1 M[Na.sup.+], 20 mM EDTA, 0.05% Tween 20). Subsequent washing and staining of the arrays was carried out using the GeneChip® fluidics station protocol EukGE_WS2v4. The EukGE_WS2v4 protocol included two post hybridization washes, staining, three post staining washes, and a final wash. The first wash consisted of 10 cycles of 2 mixes per cycle with Non Stringent Wash Buffer (6×SSPE, 0.01% Tween 20) at 25° C. The second wash consisted of 4 cycles of 15 mixes per cycle with Stringent Wash Buffer (100 mm MES, 01M [Na.sup.+], 0.01% Tween 20) at 50° C. The probe arrays were stained for 10 minutes in streptavidin-phycoerythrin solution (SAPE) (2× Stain Buffer, deionized water, 1 mg/ml SAPE (Molecular Probes, Eugene, Oreg.) 50 mg/ml BSA (Sigma, St. Louis, Mo.) at 25° C. The first post stain wash consisted of 10 cycles of 4 mixes per cycle in Non-Stringent Wash Buffer at 25° C. The second post stain wash consisted of washing probe arrays were treated for 10 minutes in antibody solution (2× Stain Buffer, 50 mg/ml BSA, 10 mg/ml normal goat IgG (Sigma Chemical, St. Louis Mo.), 0.5 mg/ml biotinylated antibody (goat), deionized water at 25° C. A third post stain wash consisted of 10 minutes in SAPE solution (2× Stain Buffer, deionized water, 1 mg/ml SAPE (Molecular Probes, Eugene, Oreg.) 50 mg/ml BSA (Sigma, St. Louis, Mo.) at 25° C. The final wash consisted of 15 cycles of 4 mixes per cycle at 30° C. in Non-Stringent Wash Buffer. Following washing and staining, probe arrays were scanned 2 times (multiple image scan) at 3 um resolution using the GeneChip® System confocal scanner made for Affymetrix.

3. Probe Arrays

The arrays were synthesized using light-directed combinatorial chemistry as described previously. The Hu6.8K_all GeneChip® probe arrays used for the current study contain probe sets representing 7129 genes. The oligonucleotides are 25 bases in length. Probes are complementary and correspond to human genes registered in Unigene, GenBank and The Institute for Genomic Research Database (TIGR). Each probe set has oligonucleotides that are identical to sequence in the gene and oligonucleotides that contain a homomeric (base transversion) mismatch at the central base position of the oligomer used for measuring cross hybridization. Probes are selected with a bias toward the 3′ region of each gene. Probe pairs representing human genes such as GAPDH, B-actin, transferrin receptor and transcription factor ISGF-3 serve as internal controls for monitoring RNA integrity. In addition, the probe arrays contain oligonucleotides representing sequences of bacterial genes, BioB, BioC, BioD, and one phage gene, Cre, as quantitative standards. Copy numbers are determined by correlating the known concentrations of the spiked standards with their hybridization. Copies per cell are calculated based on the assumption that the average transcript length is 1 kb and there are 300,000 transcripts per cell.

4. Analysis

All samples were hybridized in duplicate and only those transcripts detected as present in duplicate hybridizations or absent in duplicate hybridizations are reported. Of the transcripts present in duplicate hybridizations the hybridization values were within two fold. The values from the duplicate hybridizations were averaged. GeneChip® 3.0 software was used to scan and analyze the data. Microsoft Excel and Microsoft Access were also used for data analysis.

### CONCLUSION

The present invention provides methods and compositions for obtaining expected concentrations for a plurality of housekeeping genes and for obtaining a normalized concentration for a target in an experimental sample. It is to be understood that the above description is intended to be illustrative and not restrictive. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled. All references cited in this application are incorporated by reference for all purposes.

What is claimed is:

1. A method for obtaining an expected concentration for each of a plurality of housekeeping genes comprising:
(a) obtaining a nucleic acid sample obtained from a biological sample;
(b) obtaining a set of external transcripts wherein said set comprises a plurality of different external transcripts each present at different known concentrations;
(c) adding an aliquot of said external transcripts to said biological sample to generate a spiked sample;
(d) amplifying said spiked sample to generate an amplified sample;
(e) labeling said amplified sample to generate a labeled sample;
(f) hybridizing said labeled sample to an array to obtain a hybridization pattern;
(g) analyzing said hybridization pattern to obtain an intensity value for each external transcript and for each of said housekeeping genes;
(h) preparing a standard calibration curve with said intensity values of said external transcripts on a first axis and concentrations of said external transcripts on a second axis;
(i) fitting a curve through a plurality of points of said standard calibration curve;
(j) plotting an intensity value for each housekeeping gene onto said standard calibration curve;
(k) calculating an estimated concentration for each housekeeping gene from said standard calibration curve;
(l) repeating steps (a) through (k) for a plurality of biological samples; and
(m) combining said estimated concentrations for each housekeeping gene to obtain an expected concentration for each housekeeping gene.

2. A method according to claim 1, wherein said nucleic acid sample is a RNA.

3. A method according to claim 1, wherein said nucleic acid sample is a DNA.

4. A method according to claim 2, wherein said RNA is a poly(A) RNA.

5. A method according to claim 4, wherein said poly(A) RNA is from a human.

6. A method according to claim 1, wherein said biological sample is from a tissue sample.

7. A method according to claim 1, wherein said plurality of biological samples are all from a same organism.

8. A method according to claim 1, wherein said plurality of biological samples are all from a same organ.

9. A method according to claim 1, wherein said plurality of biological samples are all from a same gender.

10. A method according to claim 1, wherein said external transcripts are RNA.

11. A method according to claim 1, wherein between 2 and 15 external transcripts are used.

12. A method according to claim 1, wherein the concentration range of the external transcripts starts from below a detection limit and ends at a saturation point.

13. A method according to claim 1, wherein said concentration range is in attomoles/μg.

14. A method according to claim 1, wherein said concentration range is in μmoles/μg.

15. A method according to claim 1, wherein said concentration range of said spiked sample ranges from 0.1-130 attomoles/1 μg RNA spiked.

16. A method according to claim 1, wherein at least one of said external transcripts comprises yeast intergenic region (YIR) mRNAs.

17. A method according to claim 1, wherein at least one of said external transcripts comprises bacterial mRNAs.

18. A method for obtaining a normalized concentration for a target in an experimental sample comprising:
(a) obtaining a nucleic acid sample obtained from said experimental sample;
(b) obtaining a set of external transcripts wherein said set comprises a plurality of different external transcripts each present at different concentrations within a range of concentrations;
(c) adding an aliquot of said external transcripts to said experimental sample to generate a spiked sample;
(d) amplifying said spiked sample to generate an amplified sample;
(e) labeling said amplified sample to generate a labeled sample;
(f) hybridizing said labeled sample to an array to obtain a hybridization pattern;
(g) analyzing said hybridization pattern to obtain an intensity value for each external transcript and for each of said housekeeping genes;
(h) preparing an experimental calibration curve with said intensity values of said external transcripts on a first axis and concentrations of said external transcripts on a second axis;
(i) fitting a line through a plurality of points of said experimental calibration curve;
(j) plotting said intensity values from each housekeeping gene onto said experimental calibration curve;
(k) calculating a preliminary concentration for each said housekeeping gene;
(l) determining a ratio of expected concentration of said housekeeping gene from said experimental sample to said preliminary concentration for each housekeeping gene and averaging a plurality of said ratios to obtain a correction factor;
(m) plotting said intensity value for said target onto said second calibration curve to obtain a preliminary concentration for said target in said experimental sample; and
(n) multiplying said preliminary concentration for said target by said correction factor to obtain a normalized concentration for said target in said experimental sample.

19. A method according to claim 18, wherein said nucleic acid sample is selected from the group consisting of total RNA, cDNA, and poly(A) RNA.

20. A method according to claim 18, wherein 2 to 15 external transcripts are used and wherein each is present at a different concentration within said range of concentrations.

21. A method according to claim 18, wherein the lower limit of said range of concentrations is below a detection limit and the upper limit of said range of concentrations is at or above a saturation point.

22. A method according to claim 18, wherein said experimental calibration curve is non-linear.

23. A method according to claim 18, wherein said correction factor is at least 0.01.

24. A computer software product for obtaining a normalized concentration for a target in an experimental sample comprising:
(a) computer program code for preparing an experimental calibration curve with intensity values of external transcripts on a first axis and concentrations of said external transcripts on a second axis;
(b) computer program code for fitting a line through a plurality of points of said experimental calibration curve;
(c) computer program code for plotting said intensity values from each housekeeping gene onto said experimental calibration curve;
(d) computer program code for calculating a preliminary concentration for each said housekeeping gene;
(e) computer program code for determining a ratio of expected concentration of said housekeeping gene from said experimental sample to said preliminary concentration for each housekeeping gene and averaging a plurality of said ratios to obtain a correction factor;
(f) computer program code for plotting said intensity value for said target onto said second calibration curve to obtain a preliminary concentration for said target in said experimental sample; and
(g) computer program code for multiplying said preliminary concentration for said target by said correction factor to obtain a normalized concentration for said target in said experimental sample.

25. A computer software product according to claim 24, wherein said correction factor is at least 0.01.

26. A computer software product according to claim 24 wherein the curve is non-linear.

27. A computer readable medium having computer executable instructions for obtaining a normalized concentration for a target in an experimental sample comprising instructions for:
(a) preparing an experimental calibration curve with intensity values of external transcripts on a first axis and concentrations of said external transcripts on a second axis;
(b) fitting a line through a plurality of points of said experimental calibration curve;
(c) fitting said intensity values from each housekeeping gene to said experimental calibration curve;
(d) calculating a preliminary concentration for each said housekeeping gene;
(e) determining a ratio of expected concentration of said housekeeping gene from said experimental sample to said preliminary concentration for each housekeeping gene and averaging a plurality of said ratios to obtain a correction factor;
(f) plotting said intensity value for said target onto said second calibration curve to obtain a preliminary concentration for said target in said experimental sample; and
(g) multiplying said preliminary concentration for said target by said correction factor to obtain a normalized concentration for said target in said experimental sample.

28. A computer readable medium according to claim 27, wherein said experimental calibration curve is non-linear

29. A computer readable medium according to claim 27, wherein said correction factor is a mean of all ratios of expected concentration to preliminary concentration.

30. A computer readable medium according to claim 27, wherein said correction factor is a median of all ratios of expected concentration to preliminary concentration.

31. A computer readable medium according to claim 27, wherein said correction factor is at least 0.01.
